(19) United States
(12) Patent Application Publication
(10) Pub. No.: US 2009/0240218 A1
(43) Pub. Date: Sep. 24, 2009

(54) Wound Therapy System

(75) Inventors: Richard M. Braga, North Easton, MA (US); Ronald F. Vitaris, Worcester, MA (US); Shane Cooke, Wrentham, MA (US); David G. Heagle, Taunton, MA (US); Michael D. Hudspeth, Arnold, MO (US); Amaz Malhi, Watertown, MA (US); Hansen Swaniker, Bristol, RI (US); Kristin L. Watson, North Attleboro, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/407,396

(22) Filed: Mar. 19, 2009

Related U.S. Application Data
(60) Provisional application No. 61/038,301, filed on Mar. 20, 2008.
(60) Provisional application No. 61/052,007, filed on May 9, 2008.
(60) Provisional application No. 61/060,869, filed on Jun. 12, 2008.
(60) Provisional application No. 61/146,051, filed on Jan. 21, 2009.

(51) Int. Cl. A61M 1/00; A61F 13/00

(52) U.S. Cl. 604/313

(57) ABSTRACT

A portable system for subatmospheric pressure therapy in connection with healing a surgical wound includes a wound dressing dimensioned for positioning relative to a wound bed of a subject and a portable subatmospheric pressure mechanism dimensioned to be carried or worn by the subject. The subatmospheric pressure mechanism includes a housing having a control unit, a collection canister, and means for releasably connecting the housing and the canister. The collection canister collects exudates from the wound bed which has been removed under subatmospheric pressure supplied by the control unit. The connection means between the housing and the canister permits the removal of the canister for emptying thereof or replacement with a new collection canister.

### CROSS-REFERENCE TO RELATED APPLICATIONS

This application claims priority to, and the benefit of, U.S. Provisional Patent Application No. 61/038,301, filed Mar. 20, 2008, U.S. Provisional Patent Application No. 61/052,007, filed May 9, 2008, U.S. Provisional Patent Application No. 61/060,869, filed Jun. 12, 2008 and U.S. Provisional Patent Application No. 61/146,051, filed Jan. 21, 2009, the entire contents of each reference being incorporated herein by reference.

### BACKGROUND

1. Technical Field

The present disclosure relates to treating an open wound, and, more specifically, relates to a portable wound therapy system.

2. Description of Related Art

Wound closure involves the migration of epithelial and subcutaneous tissue adjacent the wound towards the center and away from the base of the wound until the wound closes. Unfortunately, closure is difficult with large wounds, chronic wounds or wounds that have become infected. In such wounds, a zone of stasis (i.e. an area in which localized swelling of tissue restricts the flow of blood to the tissues) forms near the surface of the wound. Without sufficient blood flow, the epithelial and subcutaneous tissues surrounding the wound not only receive diminished oxygen and nutrients, but, are also less able to successfully fight microbial infection and, thus, are less able to close the wound naturally. Such wounds have presented difficulties to medical personnel for many years.

Negative pressure therapy also known as suction or vacuum therapy has been used in treating and healing wounds. Application of negative pressure, e.g. reduced or subatmospheric pressure, to a localized reservoir over a wound has been found to assist in closing the wound by promoting blood flow to the area, stimulating the formation of granulation tissue, and encouraging the migration of healthy tissue over the wound. Negative pressure may also inhibit bacterial growth by drawing fluids from the wound such as exudates, which may tend to harbor bacteria. This technique has proven particularly effective for chronic or healing-resistant wounds, and is also used for other purposes such as post-operative wound care.

Generally, negative pressure therapy provides for a wound to be covered to facilitate suction at the wound area. A conduit is introduced through the wound covering to provide fluid communication to an external vacuum source. Atmospheric gas, wound exudates, or other fluids may thus be drawn from the reservoir through the fluid conduit to stimulate healing of the wound. Exudates drawn from the reservoir may be deposited in a collection canister. The various components of the wound therapy system may need to be disconnected or be replaced for a variety of reasons, such as component failure or different component life expectancies. It would be advantageous to provide a user friendly mechanism for connecting and disconnecting components of the system.

### SUMMARY

Accordingly, a portable system for subatmospheric pressure therapy is utilized in connection with healing a surgical wound. The system includes a wound dressing dimensioned for positioning relative to a wound bed of a subject and a portable subatmospheric pressure mechanism dimensioned to be carried or worn by the subject. The subatmospheric pressure mechanism includes a housing having a control unit, a collection canister, and means for releasably connecting the housing and the canister.

Various embodiments of means for releasably coupling the housing and the canister are disclosed, including latch couplings, bayonet mounts, fastening mechanisms, and magnetic couplings. The connection means may facilitate the connection, disconnection, or maintenance of components of the system including the replacement of the collection canister. The connection means permits the collection canister to be released for emptying or disposal during a course of therapy. In one method of application, the collection canister collects exudates from the wound bed which has been removed under subatmospheric pressure supplied by the control unit. When full, the collection canister is removed and replaced with a new canister which is also connectable through connection means with the housing of the control unit. The control unit may be intended for reuse.

### BRIEF DESCRIPTION OF THE DRAWINGS

Various embodiments of the wound dressing system of the present disclosure are described herein with reference to the drawings wherein:

FIG. 1A is a view of the portable wound therapy system of the present disclosure illustrating the wound dressing in cross-section and the subatmospheric pressure mechanism;

FIG. 1B is a cross-sectional view of a subatmospheric pressure mechanism in accordance with the present disclosure;

FIG. 2A is a perspective view of the subatmospheric pressure mechanism illustrating the control unit housing, collection canister and a latch coupling for releasably connecting the collection canister to the control unit housing;

FIG. 2B is a side plan view of the latch coupling of FIG. 2A;

FIG. 2C is a perspective view of another embodiment of a latch coupling mechanism for releasably connecting the control unit housing and the collection canister;

FIG. 2D is a side plan view of the latch coupling mechanism of FIG. 2C;

FIG. 3A is a perspective view of another embodiment of a latch coupling mechanism for releasably connecting the control unit housing and the collection canister;

FIG. 3B is a perspective view of another embodiment of a latch coupling mechanism of FIG. 3A;

FIGS. 4A-4D are perspective views of other alternative embodiments of a latch coupling mechanism for releasably connecting the control unit housing and the collection canister;

FIG. 5 is a perspective view of the collection canister including a second coupling mechanism utilizing a tongue;

FIG. 6A is a cross-sectional view of another embodiment of a latch coupling mechanism for releasably connecting the control unit housing and the collection canister;

FIG. 6B is a cross-sectional view of the latch coupling mechanism of FIG. 6A in a disengaged position;

FIG. 7A is a cross-sectional view of another embodiment of a latch coupling mechanism for releasably coupling the control unit housing and the collection canister and depicted in an engaged position;

FIG. 7B is a cross-sectional view of the latch coupling of FIG. 7A in a disengaged position;

FIG. 8A is a view of a carrier support apparatus for supporting components of the subatmospheric pressure mechanism;

FIG. 8B is a view of an alternate carrier support apparatus for supporting components of the subatmospheric pressure mechanism of the present disclosure;

FIG. 9 is a perspective view illustrating one embodiment of a bayonet mount for releasably mounting the control unit housing and the collection canister of the subatmospheric pressure mechanism;

FIGS. 10A-10C are partial cross-sectional views illustrating a sequence of operation for connecting the collection canister and the control unit housing with the bayonet mount of FIG. 9;

FIG. 11 is a perspective view illustrating another embodiment of a bayonet mount for releasably mounting the control unit control unit housing and the collection canister of the subatmospheric pressure mechanism;

FIGS. 12A-12C are partial cross-sectional views illustrating a sequence of operation for connecting the collection canister and the control unit housing with the bayonet mount of FIG. 11;

FIGS. 13-15B are views of different embodiments of the subatmospheric pressure mechanism having strap fasteners;

FIGS. 16A-21B are views of different embodiments of the subatmospheric pressure mechanism having complementary fastening elements on the periphery of the housing and the canister;

FIGS. 22A and 22B are views of an embodiment of the subatmospheric pressure mechanism having spring-loaded button fasteners in an uncoupled and coupled state, respectively;

FIGS. 23 and 24 are views of alternate embodiments of a subatmospheric pressure mechanism having a spring-loaded button fastener;

FIGS. 25 and 26 are views of different embodiments of the subatmospheric pressure mechanism having a slide fastener;

FIGS. 27A-28 are views of alternate embodiments of a subatmospheric pressure mechanism each having a spring-assisted clip fastener;

FIGS. 29A and 29B are view of an embodiment of the subatmospheric pressure mechanism having movable clip fasteners in an uncoupled and coupled state;

FIG. 30A is a cross-sectional view of a magnetic coupling of the control unit and the collection canister of the subatmospheric pressure mechanism;

FIG. 30B is a cross-sectional view of an alternate magnetic coupling arrangement of the control unit and collection canister;

FIG. 30C is a cross-sectional view of another alternate magnetic coupling arrangement of the control unit and collection canister;

FIGS. 30D-30F are top plan views of different magnet configurations within the control unit; and

FIG. 31 is a cross-sectional view of a magnetic coupling of the control unit and the collection canister via use of permanent magnets.

### DESCRIPTION OF THE EMBODIMENTS

The wound therapy system of the present disclosure promotes healing of a wound via the use of a wound dressing and a subatmospheric pressure mechanism. Generally, the subatmospheric pressure mechanism applies subatmospheric pressure to the wound to effectively remove wound fluids or exudates captured by the composite wound dressing, and to increase blood flow to the wound bed and enhance cellular stimulation of epithelial and subcutaneous tissue. The wound therapy system may be entirely portable, i.e., it may be worn or carried by the subject such that the subject may be completely ambulatory during the therapy period. The wound therapy system including the subatmospheric pressure mechanism and components thereof may be entirely disposable after a predetermined period of use or may be individually disposable whereby some of the components are reused for a subsequent therapy application.

The wound therapy system of the present disclosure promotes healing of a wound in conjunction with subatmospheric negative pressure therapy. The system may incorporate a variety of wound dressings, subatmospheric pressure sources and pumps, and collection canisters.

The attached figures illustrate exemplary embodiments of the present disclosure and are referenced to describe the embodiments depicted therein. Hereinafter, the disclosure will be described by explaining the figures wherein like reference numerals represent like parts throughout the several views.

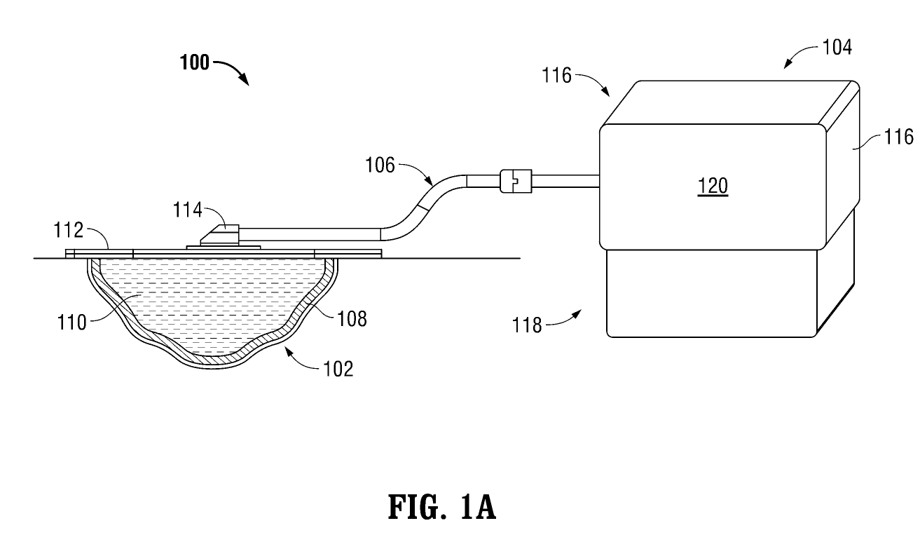

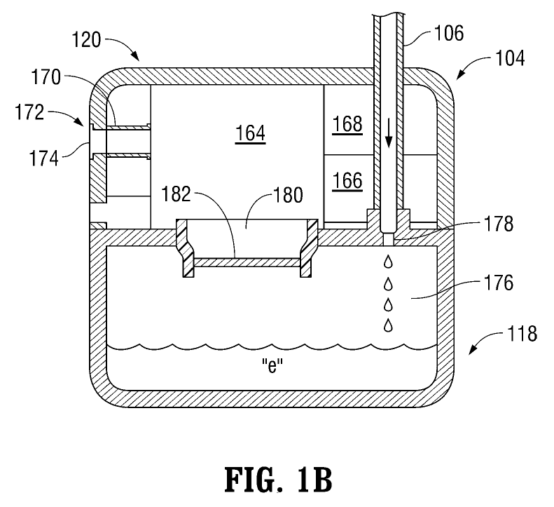

Referring initially to FIG. 1A, wound therapy system 100 according to the present disclosure is illustrated. Wound therapy system 100 includes composite wound dressing 102 and subatmospheric pressure mechanism 104 in fluid communication with the wound dressing 102 through conduit 106.

Wound dressing 102 may include several components, namely, wound contact layer or member 108, a wound packing member or filler 110 supported by the contact member 108 and outer layer or cover member 112. Wound contact member 108 is adapted to substantially conform to the topography of a wound bed “w.” Wound contact member 108 is substantially porous or perforated to permit exudates to pass from the wound bed “w” through the wound contact member 108. The passage of wound exudates through the wound contact member 108 may be unidirectional such that wound exudates do not flow back to the wound bed “w.” Unidirectional flow may be encouraged by directional apertures formed in contact member 108 or a lamination of materials having absorption properties differing from those of contact member 108. A non-adherent material may be selected such that contact member 108 does not tend to cling to wound bed “w” or surrounding material when it is removed. Exemplary materials that may be used as a contact member 108 are sold under the trademarks CURITY® Non-Adherent Dressing or XEROFLO® by Tyco Healthcare Group, LP (d/b/a Covidien)

Wound packing member 110 of wound dressing 102 is intended to absorb and transfer wound fluid and exudates. Wound packing member 110 is conformable to assume the shape of any wound bed “w.” Wound packing member 110 may be treated with agents such as polyhexamethylene biguanide (PHMB) to decrease the incidence of infection, or other medicants to promote healing of the wound. A suitable wound packing material 110 is the antimicrobial dressing sold under the trademark KERLIX™ by Tyco Healthcare Group, LP (d/b/a Covidien).

Outer member or wound covering 112 encompasses the perimeter of the wound dressing 102 to surround wound bed “w” and to provide a liquid-tight seal around the perimeter “p” of the wound bed “w.” For instance, the sealing mechanism may be any biocompatible adhesive bonded to the perimeter of wound covering 112. Thus, wound covering 112 may act as both a microbial barrier and a fluid barrier to prevent contaminants from entering wound bed “w” and for maintaining the integrity thereof.

Wound covering 112 is typically a flexible material, e.g., resilient or elastomeric, that seals the top of wound dressing 102 to prevent passage of liquids or contamination to and from the wound dressing 102. Wound covering 112 may be formed from a moisture vapor permeable membrane to promote the exchange of oxygen moisture between the wound bed “w” and the atmosphere. A membrane that provides a sufficient moisture vapor transmission rate is a transparent membrane sold under the trade name POLYSKIN® II by Tyco Healthcare Group, LP (d/b/a Covidien). A transparent membrane permits an assessment of wound conditions to be made without requiring removal of the wound covering 112. Alternatively, wound covering 112 may comprise an impermeable membrane or a substantially rigid membrane.

Wound covering 112 may include a port or connector 114 in fluid communication with the interior of wound dressing 102 to facilitate connection of wound dressing 102 to conduit or tubing 106. Conduit 106 defines a fluid flow path leading through wound therapy system 100. Connector 114 may be a rigid or flexible, low-profile component, and may be adapted to receive conduit 106 in a releasable and fluid tight manner. A hollow interior of connector 114 provides fluid communication between conduit 106 and the interior of wound dressing 102. Connector 114 may have a valve built (not shown), e.g., a one-way valve to permit exudates to flow in one direction only, i.e., away from wound dressing 102 toward subatmospheric pressure mechanism 104. Connector 114 may be provided as a pre-affixed component of wound dressing 102, as a component of conduit 106 or entirely separate and connected thereto by conventional means. Alternatively, connector 114 may be eliminated if other provisions are made for providing fluid communication between wound dressing 102 and conduit 106.

Conduit 106 extends from subatmospheric pressure mechanism 104 to provide fluid communication between the interior of the wound dressing 102 and subatmospheric pressure mechanism 104. Any suitable conduit may be used including those fabricated from flexible elastomeric or polymeric materials. Conduit 106 may connect to subatmospheric pressure mechanism 104 or other system components by conventional air tight means such as friction fit, bayonet coupling, or barbed connectors. The conduit connections may be made permanent, or alternatively a quick-disconnect or other releasable means may be used to provide some adjustment flexibility to the apparatus. Fluid conduit 106 may comprise the same material of construction along the entire length of the tubing or may assume an alternate form, e.g., it may include several distinct tubes connected to each other through conventional means.

Subatmospheric pressure mechanism 104 will be discussed subatmospheric pressure mechanism 104 includes control unit 116 and collection canister 118. Control unit 116 has control unit housing 120 which houses the software, logic and components required to operate the subatmospheric pressure mechanism 104.

Referring now to FIG. 1B, the electronic, electrical and pneumatic components of the subatmospheric pressure mechanism 104 will be discussed. Subatmospheric pressure mechanism 104 may incorporate vacuum source or pump 164, actuator or motor 166 for activating the vacuum source 164, and power source 168. Vacuum source or pump 164 generates or otherwise provides negative pressure to wound therapy system 100. Vacuum source or pump 164 may be a pump of the diaphragmatic, peristaltic or bellows type or the like, in which the moving part(s) draw exudates out of the wound bed “w” into the wound dressing 102 by creating areas or zones of decreased pressure e.g., vacuum zones with the wound dressing 102. This area of decreased pressure preferably communicates with the wound bed “w” to facilitate removal of the fluids therefrom and into the absorbent or non-absorbent packing member 110.

Vacuum source or pump 164 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg and about 125 mmHg, or between about 30 mmHg and 80 mmHg. Vacuum source or pump 164 is actuated by actuator 166 which may be any means known by those skilled in the art, including, for example, AC motors, DC motors, voice coil actuators, solenoids, and the like. Actuator 166 may be incorporated within pump 164.

Power source 168 may be disposed within housing 120 or separately mountable to housing 120. A suitable power source 168 includes alkaline batteries, wet cell batteries, dry cell batteries, nickel cadmium batteries, solar generated means, lithium batteries, NiMH batteries (nickel metal hydride) each of which may be of the disposable or rechargeable variety.

Housing 120 may further include vent portal 170 configured to vent exhaust air from vacuum source or pump 164 through exhaust port 172. Vent portal 170 extends from housing 120 and may be directly connected to vacuum source 164. It is also envisioned that vent portal 170 may exhaust air from within housing 120 rather than directly from vacuum source 164. Exhaust port 172 may include filter 174 extending across the exhaust port 172. Filter 174 may be a bacterial filter to prevent emission of bacteria from housing 120. A PCB may be provided along with a pressure transducer to control output of the pump in response to pressure measurements calculated in the collection canister.

Collection canister 118 collects exudates “e” removed from the wound bed “w” during therapy through conduit or tubing 106. A fluid inlet 178 and suction port 180 may be maintained between the housing 120 and the canister 118. Fluid inlet 178 is configured to operably engage conduit 106. Fluid inlet 178 may be connectable with conduit 106 by conventional air and fluid tight means, such as those described above. In embodiments, fluid inlet 178 may contain a luer lock or other connector within the purview of those skilled in the art to secure the end of conduit 106 with the fluid inlet 178. It is envisioned that fluid inlet 178 is configured to receive a cap in order to prevent leakage of exudates and odor from internal chamber 176 of collection canister 118 when housing 120 is separated from the canister 118.

Suction port 180 is in fluid communication with vacuum source or pump 164. A filter 182, such as a hydrophobic membrane or baffling to prevent exudates from being aspirated into pump 164 may be disposed adjacent or within suction port 180. Filter 182 may also include charcoal or other odor absorbing materials and may prevent the passage of bacteria. Pump 164 creates a vacuum within internal chamber 176 of collection canister 118 by drawing air through suction port 180.

Collection canister 118 collects the exudates removed from the wound bed “w” during therapy through conduit, or tubing, 106. Collection canister 118 is releasably connected to housing 120 of control unit 116. Collection canister 118 may include any container suitable for containing wound fluids. Collection canister 118 may be substantially rigid in order to maintain the integrity and shape of the canister as a stand alone component. In the alternative, collection canister 118 may be relatively flexible and/or partly expandable to accommodate the wound exudates. Collection canister 118 may contain an absorbent material to consolidate or contain the wound drainage or debris. In other embodiments, at least a portion of collection canister 118 may be transparent to assist in evaluating the color, quality, or quantity of wound exudates. This transparency may assist in determining the remaining capacity of the canister or when the canister should be replaced.

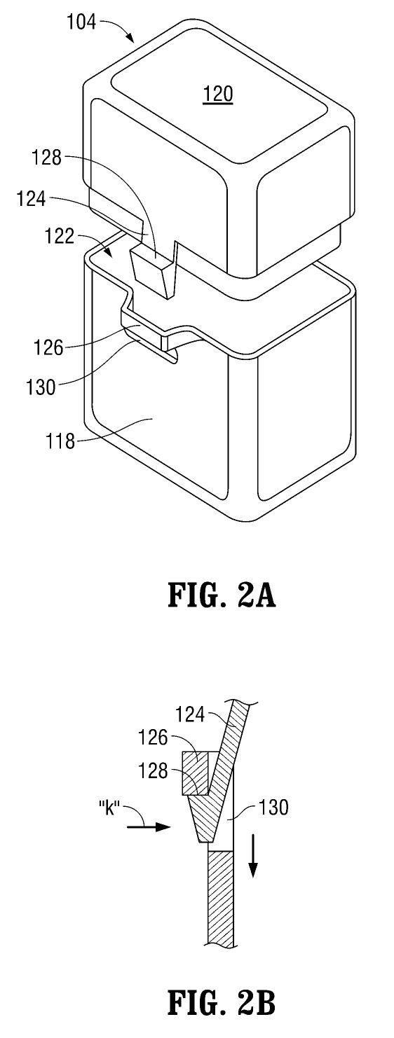

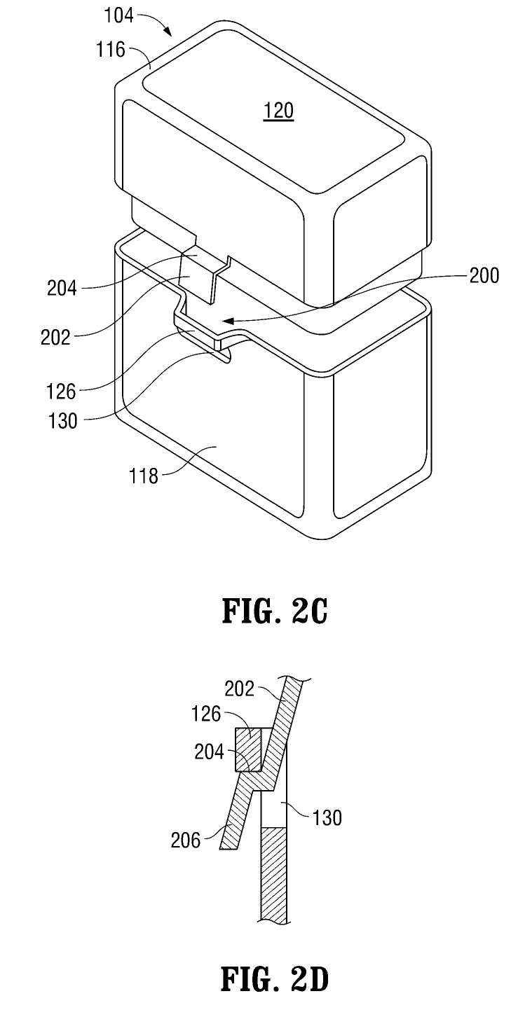

Referring now to FIG. 2A, subatmospheric pressure mechanism 104 includes a latch coupling mechanism 122 adapted for selectable releasable coupling of control unit housing 120 and collection canister 118. Latch coupling mechanism 122 may facilitate the connection, disconnection, or maintenance of components of system 100, including the replacement of collection canister 118. Latch coupling mechanism 122 includes first and second coupling segments 124, 126 associated with control unit housing 120 and collection canister 118, respectively. First coupling segment 124 may be a latch having locking surface 128. Locking surface 128 may be a curved or angular portion adapted for releasable engagement with second coupling segment. Second coupling segment 126 includes a notch or locking slot 130 for receiving latch. In the alternative, first coupling segment 124 of control unit housing 120 may be in the form of a locking slot while second coupling segment 126 of collection canister 118 may be a latch.

Latch coupling mechanism 122 is placed in the engaged position through insertion of first coupling segment or latch 124 within locking slot 130 whereby locking surface 128 engages portions of collection canister 118 defining the locking slot 130 in secured relation therewith. Latch coupling mechanism 122 is released by depressing latch 124 inwardly in the direction of directional arrow “k” of FIG. 2B toward collection canister 118 such that locking surface 128 is released from within locking slot 130. Latch 124 has sufficient flexibility to pivot out of engagement with collection canister 118 during movement to the release position. In one embodiment, latch 124 is monolithically formed with control unit housing 120. Once release is achieved, housing 120 may be lifted from collection canister 118.

FIGS. 2C and 2D illustrate another embodiment of the latch coupling mechanism. Latch coupling mechanism 200 incorporates elongated latch 202 defining a general “z-shape,” and having locking surface 204 positioned substantially at the center of the latch 202. This arrangement of latch 202 provides a manually engaging segment 206 depending from locking surface 204, which is relatively elongated and displaced from the wall of collection canister 118. The displaced orientation, in conjunction with the elongated characteristic, may provide mechanical advantages and enhance relative ease of manipulation and/or control of the latch 202 for the clinician. Latch 202 cooperates with locking slot 130 to releasably couple collection canister and control unit housing 120 in a manner similar to the embodiment of FIGS. 2A-2B.

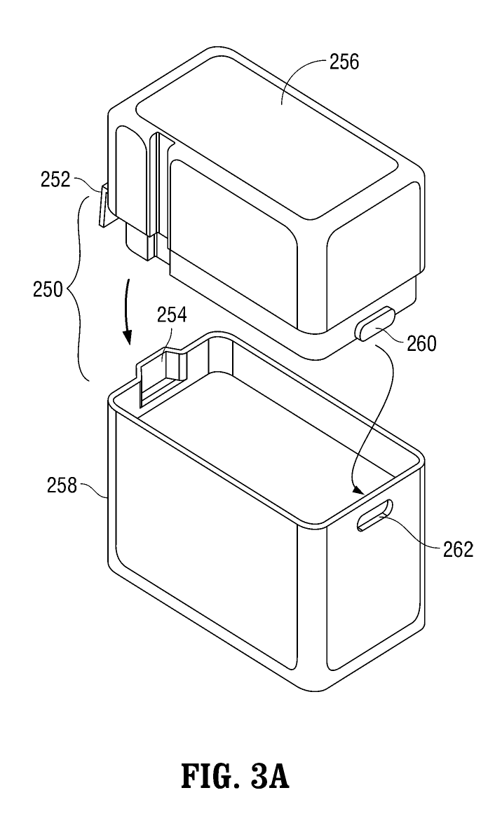

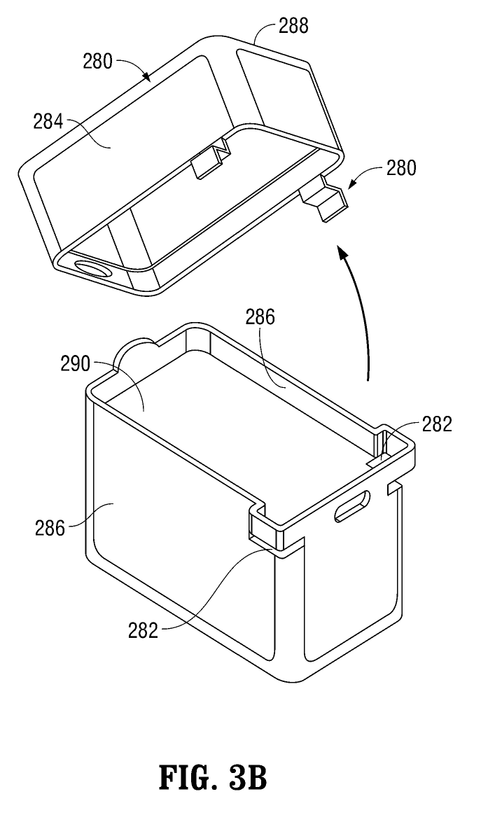

FIG. 3A illustrates an alternate embodiment of the latch coupling mechanism of the present disclosure. Latch coupling mechanism 250 includes latch 252 and associated locking slot 254 disposed at respective longitudinal ends of control unit housing 256 and collection canister 258, respectively. Latch 252 and locking slot 254 function in a manner similar to the latch coupling mechanism of FIGS. 2A and 2B. In addition, control unit housing 256 includes outwardly depending tab 260 adjacent the other longitudinal end of the control unit housing 256. Tab 260 is received within a corresponding positioned and dimensioned tab slot 262 defined in collection canister 258. Control unit housing 256 may be mounted to collection canister 258 by pivoting the control unit housing 256 relative to the collection canister 258 in a manner to position tab 260 within tab slot 262. Thereafter, control unit housing 256 is pivoted about the longitudinal end containing tab 260 to insert latch 252 within locking slot 254 until the latch 252 is secured within the locking slot 254. Release of control unit housing 256 is effected by depressing latch 252 inwardly to be released from locking slot 254, and, thereafter, pivoting the control unit housing 256 about tab 260 to remove the tab 260 from tab slot 262.

FIG. 3B illustrates an alternate embodiment including a pair of latches 280 and associated slots 282 defined within the side walls 284, 286 of control unit housing 288 and collection canister 290. In other respects, this embodiment is substantially similar to the embodiment of FIG. 3A.

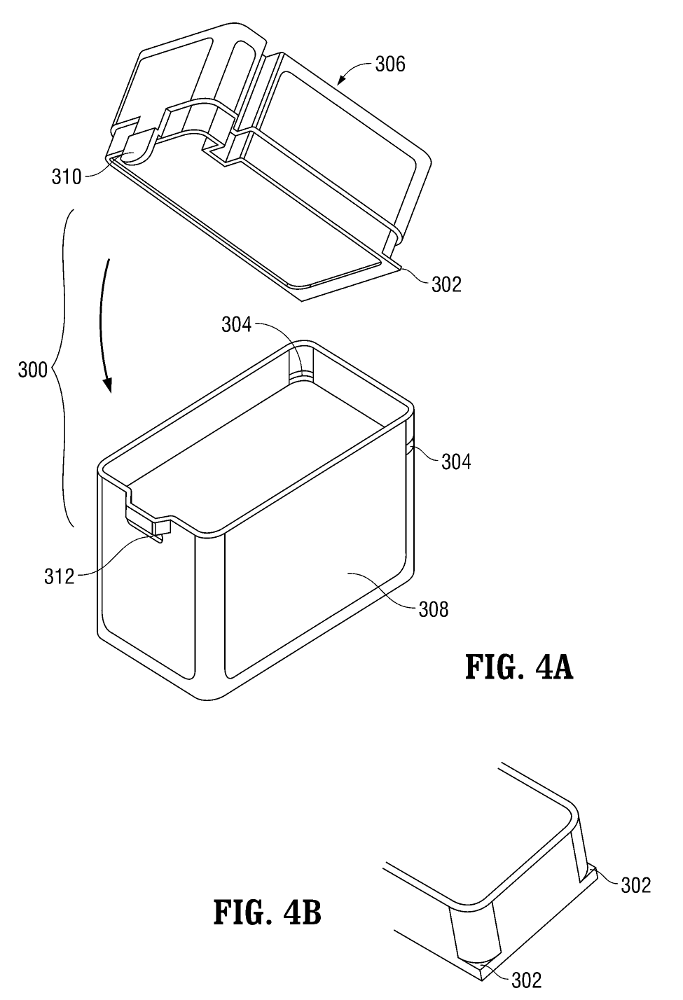

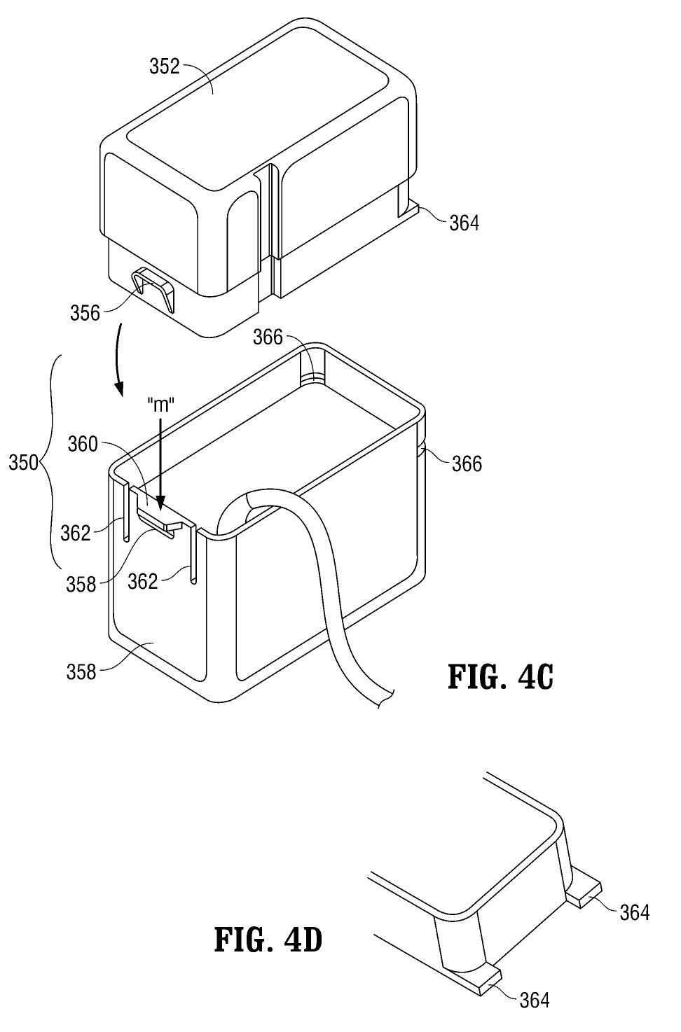

FIGS. 4A and 4B illustrate an alternate embodiment of the latch coupling mechanism of the present disclosure. Latch coupling mechanism 300 incorporates a pair of tabs 302 and associated tab slots 304 at the longitudinal ends of control unit housing 306 and collection canister 308, respectively. Tabs 302 and slots 304 may be disposed adjacent respective corners to establish a pivot axis for rotating control unit housing 306 relative to collection canister 308 during release and securement of the two components. Latch coupling mechanism 300 further includes locking latch 310 and associated locking slot 312 at the other longitudinal end of control unit housing 306 and collection canister 308, respectively. Locking latch 310 and locking slot 312 function in a manner similar to the embodiments of FIGS. 2A-2D.

FIGS. 4C and 4D illustrate an alternate embodiment of latch coupling mechanism 350. Control unit housing 352 and collection canister 354 include latch 356 and locking slot 358, respectively. Collection canister 354 further includes release tab 360 adjacent locking slot 358 and a pair of relief grooves or slots 362 on each side of the locking slot 358. Latch 356 is received within locking slot 358 when in the engaged position of collection canister 354 and control unit housing 352. To release the components, release tab 360 may be depressed in a downward direction “m” indicated in FIG. 4C to cause the release tab 360 to pivot outwardly through an angular range of movement and displace locking slot 358 from latch 356. Such movement of release tab 360 relative to collection canister 354 is facilitated by relief grooves 362. Upon release of locking slot 358 from latch 356, control unit housing 352 is removed from collection canister 354. Latch coupling mechanism may further include a pair of tabs 364 and associated tab slots 366 at the longitudinal ends of control unit housing 352 and collection canister 354, respectively.

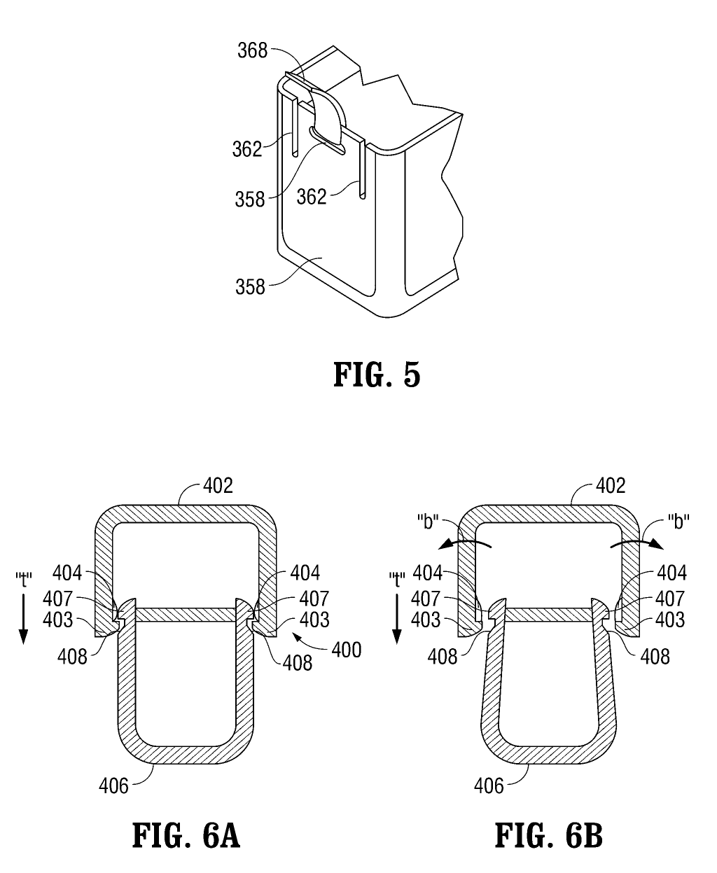

Referring now to FIG. 5, an alternate embodiment of latch coupling mechanism 350 incorporates release tongue 368 in lieu of a release tab 360. Release tongue 368 may be a strip, band, or section of material looped around locking slot 358. When latch 356 is engaged with locking slot 358, the operator may pull on tongue 368 in an outward direction thereby deforming portions of collection canister adjacent locking slot 358 as facilitated by relief grooves 362, to permit release of the latch 356 from the slot 358, and subsequent release of collection canister 354 from control unit housing 352.

FIGS. 6A and 6B illustrate an alternate embodiment of latch coupling mechanism 400 in an engaged and disengaged position, respectively. Control unit housing 402 includes a pair of opposed internal locking detents 404 which change position from a locked or contracted state as depicted in FIG. 6A to an unlocked or expanded state as depicted in FIG. 6B upon external contact. Collection canister 406 incorporates a pair of locking elements 407 associated with a pair of outer locking recesses 408 in opposed relation to the locking detents 404. Locking elements 407 may be normally biased toward the radial outward position, e.g., in the direction of arrow “b” depicted in FIG. 6B. Consequently, as control unit housing 120 is positioned over and depressed or moved in the direction of directional arrow “t” in FIG. 6A, locking detents 404 contact locking elements 407 thereby locking locking detents 404 in a contracted position. With this arrangement, locking elements 407 are directed radially inwardly for reception with control until housing 120. Once positioned within control until 120, locking elements 407 are released to permit locking grips 403 to be received within locking recesses 408 to secure collection canister 406 relative to control unit housing 402.

Collection canister 406 is released from control unit housing 402 by depressing or moving the control unit housing 402 in the direction of directional arrow “t” in FIG. 6B. As the control unit housing 402 moves relative to collection canister 406, locking detents 404 contact locking elements 407 thereby unlocking the locking detents 404 to an expanded state and directing locking elements 407 radially inwardly. Locking grips 403 are now cleared from locking recesses 408. In this position, control unit housing 402 is free to be removed from collection canister 406 permitting disposal of the canister 406 and subsequent replacement with a new canister.

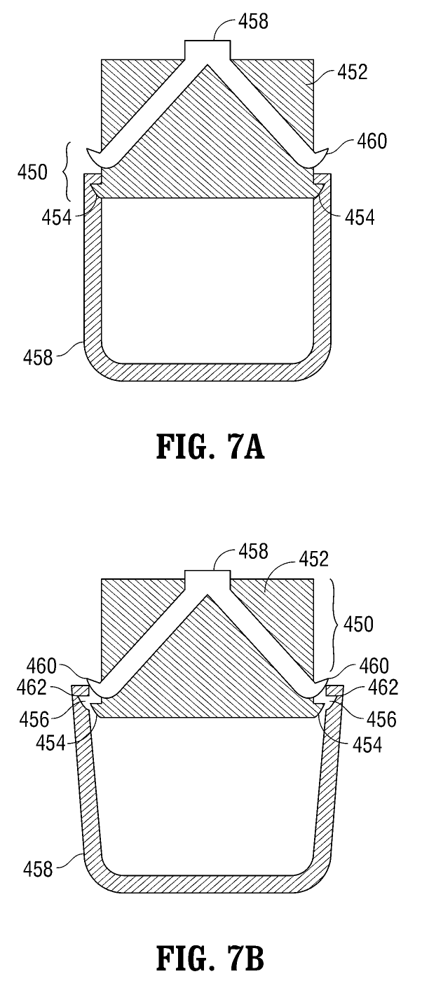

Referring now to FIGS. 7A and 7B, in an alternate embodiment, latch coupling mechanism 450 includes control unit housing 452 having two locking latches 454 disposed in diametrical opposed relation. Locking latches 454 are received within corresponding locking recesses 456 of collection canister 458 when in the engaged or secured position of control unit housing 452 depicted in FIG. 7A. Control unit housing 452 further includes release button 458 mounted to the control unit housing 452. Release button 458 includes outer cam surfaces 460 depending outwardly from control unit housing 452. Release button 458 is adapted for movement relative to control unit housing 452 from the position depicted in FIG. 7A to the position depicted in FIG. 7B. During movement of the release button 458 to the position of FIG. 7B, outer cam surfaces 460 engage internal surfaces 462 of collection canister 458 and bias the wall surfaces of the canister 458 radially outwardly as shown in FIG. 7B. In this position, locking latches 454 are released from locking recesses 456 thereby permitting removal of collection canister 458. Any means for mounting release button 458 for movement within control unit housing 452 are envisioned.

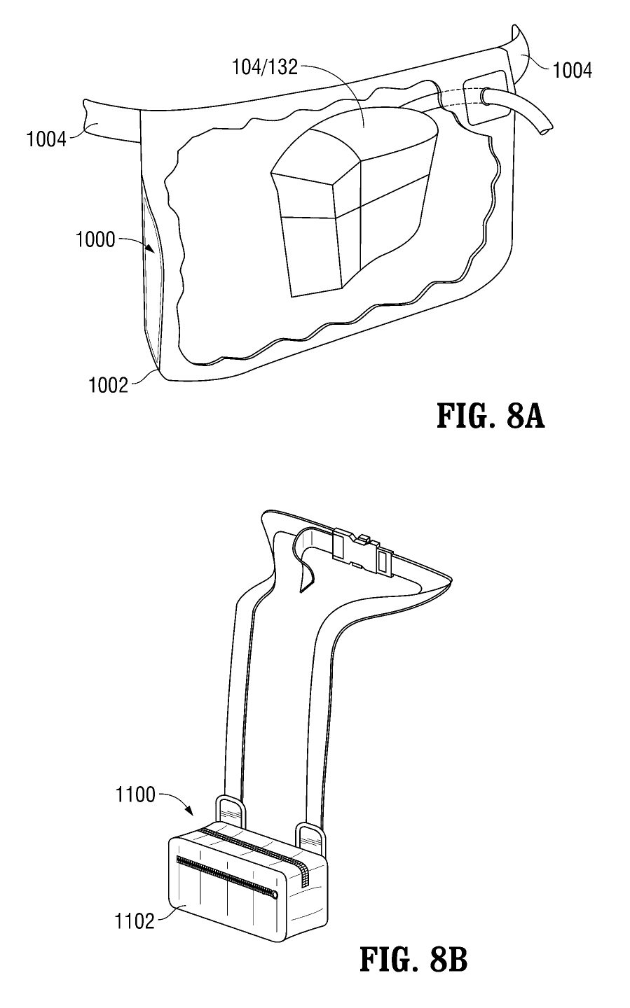

With reference now to FIG. 8A, there is illustrated a body support bag 1000 for supporting at least the subatmospheric pressure mechanism 104 and at least canister 118. As discussed, the wound therapy system 100 of the present disclosure is adapted for mounting to the body of the patient to be a self contained portal unit. In this regard, the subatmospheric pressure mechanism and canister may be at least partially carried or supported by the body support bag 1000. The body support bag 1000 generally includes a pouch 1002 and at least one strap 1004, in embodiments, two straps, for securing the pouch 1002 to the body of the patient. The body support bag 1000 is intended to receive and store at least subatmospheric pressure mechanism 104 and collection canister 118. The body support bag 1000 may be worn about the waist of the patient such as with a belt loop. This is desirable in that it may reduce the length of tubing needed depending on the location of the wound. In addition, the pouch 1002 may be located adjacent the abdomen of the patient which may present a significantly enhanced ability to conceal the system. Tubing 1006 may be secured to the body with tape, straps, or the like, or, optionally, may be unsecured and disposed beneath the patient's clothing. Thus, the body support bag 1000 permits the patient to move without restrictions or limitations, and provides an entirely portable capability to the patient during wound drainage and healing.

FIG. 8B illustrates an alternate embodiment of the body support bag. In accordance with this embodiment, the body support bag 1100 is adapted for mounting to the shoulder of the patient and has a pouch 1102. In other respects, the body support bag 1100 functions in a similar manner to the body support bag of FIG. 8A.

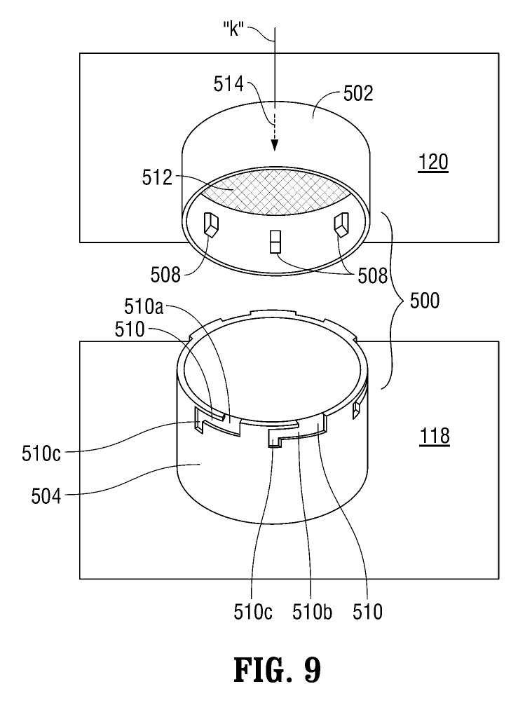

Referring now to FIG. 9, an alternate mechanism for coupling control unit housing 120 and collection canister 118 is illustrated. In this embodiment, subatmospheric pressure mechanism 104 includes a bayonet mount or coupling mechanism 500 for selectable releasable coupling and decoupling of control unit housing 120 and collection canister 118. In this manner, bayonet mount 500 may facilitate the connection, disconnection, or maintenance of components of system 100, including the replacement of collection canister 118. In one embodiment, bayonet mount 500 has first and second coupling segments 502, 504 disposed substantially centered on control unit housing 120 and canister 118, respectively. Coupling segments 502, 504 may be off-centered if desired. First and second coupling segments 502, 504 are generally cylindrical in configuration and depend from their respective component toward each other. First coupling segment 502 may define an internal dimension or diameter slightly greater than a correspondingly internal dimension or diameter of second coupling segment 504 whereby the first coupling segment 502 receives the second coupling segment 504 in the mated condition of the components. In one aspect, either coupling segment 502, 504 may incorporate a gasket or 0-ring seal (not shown) whereby a substantial seal is formed when the components are coupled. Coupling segment 502 may include at least one internal bayonet projection or bayonet lug 508 depending radially inwardly relative to an axis “k” of the first coupling segment 502. Second coupling segment 504 includes at least one correspondingly dimensioned and positioned lug retaining slot 510. In one embodiment, a plurality of bayonet lugs 508 and corresponding retaining slots 510 are radially spaced about their respective components. Each bayonet lug 508 may be a pin, knob, ball, knot, or other protrusion transverse to longitudinal axis “k.”

Lug retaining slot 510 is a generally “L”, “U” or “Z” shaped recess, through-hole, groove, or the like. For example, each lug retaining slot 510 has lug receiving segment 510a, transverse segment 510b and retention segment 510c. Bayonet mount 500 further includes a spring mechanism or resilient member 512 disposed at the intersection of first and second coupling segments 502, 504 when mated. Spring mechanism 512 may be in the form of a resilient or elastomeric washer or disc disposed within first coupling segment 502. Spring mechanism 512 facilitates retention of bayonet lugs 508 within retaining slots 510 by biasing the first coupling segment 502 away from the second coupling segment 504 when the coupling segments 502, 504 are mated as will be discussed.

First and second coupling segments 502, 504 also may include visual indicia 514 to facilitate alignment of the respective bayonet lugs 508 and retaining slots 510 of the first and second coupling segments 502, 504. Such visual indicia may be arrows, dots, lines on the exterior surfaces of first and second coupling segments 502, 504 as depicted in FIG. 9.

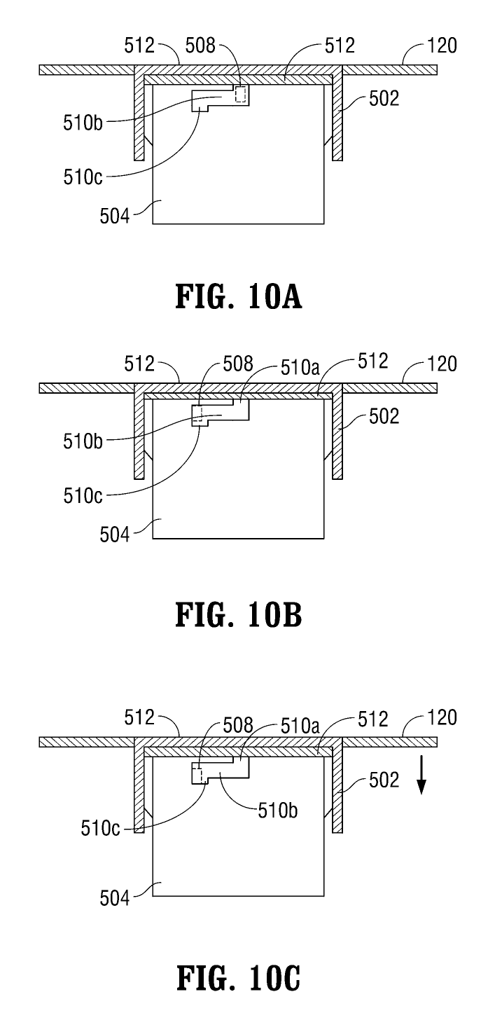

As illustrated in FIGS. 10A-10C, first coupling segment 502 is positioned over second coupling segment 504 when control unit housing 120 is aligned with canister 118. Each bayonet lug 508 enters lug receiving segment 510a of each retaining slot 510 and is advanced to be aligned with transverse segment 510b of the lug retaining slot 510. (FIG. 10A). During this movement, spring mechanism 512 is compressed as depicted in FIG. 10B. Thereafter, first and second coupling segments 502, 504 are rotated relative to each other through a predetermined angular sector of rotation whereby bayonet lug 508 is adjacent retention segment 510c. The clinician then may release at least one of the coupling segments 502, 504, which, effectively causes each bayonet lug 508 to be received within retention segment 510c of retaining slot 510 under the bias of spring mechanism 512. Spring mechanism 512 possesses sufficient resiliency to effectively secure bayonet lug(s) 508 within its respective retention segment(s) 510c of retaining slot(s) 510. FIG. 10C illustrates spring mechanism 512 expanded to secure bayonet lug 508 within retention segment 510c.

Removal of collection canister 118 from control unit housing 120 may be performed by advancing collection canister 118 relative to control unit housing 120 against the bias of spring mechanism 512 whereby each bayonet lug 508 traverses retention segment 510c to be in alignment with transverse segment 510b. Collection canister 118 and control unit housing 120 are rotated relative to each other to cause each bayonet lug 508 to be positioned in alignment with a corresponding lug receiving segment 510a of retaining slot 510. Collection canister 118 is then removed from control unit housing 120 with bayonet lug(s) 508 exiting the lug receiving segments 510a.

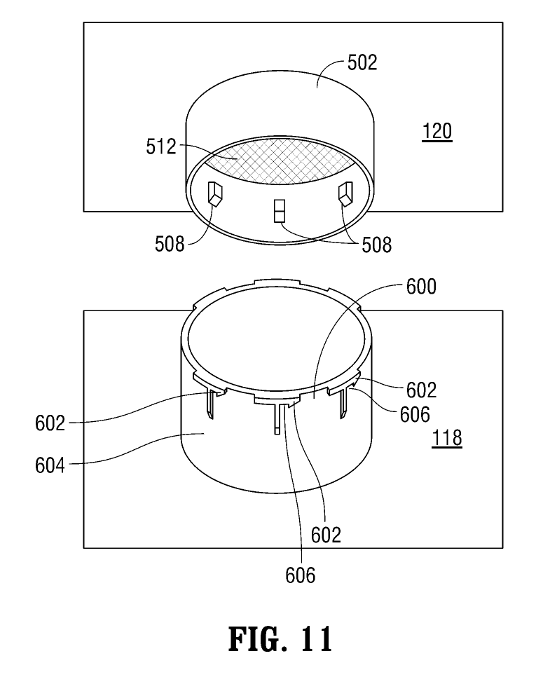

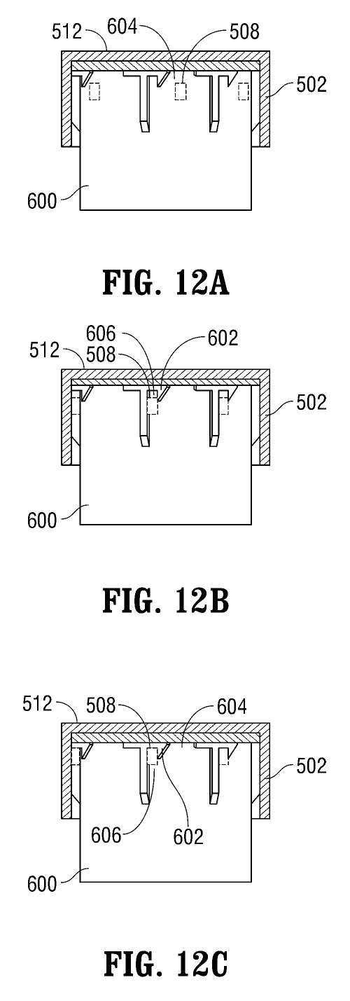

FIGS. 11 and 12A-12C illustrate an alternate embodiment of a bayonet coupling. In accordance with this embodiment, second coupling segment 600 incorporates a plurality of latches 602 to secure bayonet lugs 508. In use, bayonet lugs 508 are aligned with recesses 604 of second coupling segment 600 to position first coupling segment 502 relative to second coupling segment 600 (FIG. 12A). Thereafter, first and second coupling segments 502, 600 are rotated relative to each other whereby bayonet lugs pass 508 pass latches 602 to be received within locking recesses 606 (FIG. 12B). Latches 602 are sufficiently flexible to permit passage of bayonet lugs 508, but, return to their original position once received within locking recesses 606 (FIG. 12C). This mechanism may provide a more permanent connection. As an alternative, first coupling segment 502 may incorporate spring mechanism 512 which may be compressed to displace bayonet lugs 508 from locking recesses 606 thereby permitting relative rotational movement of the first and second coupling segments 502, 600 and removal of collection canister 118 from control unit housing 120.

Bayonet mount 500 may be constructed as shown in the exemplary embodiments above, or in reverse so that coupling segment 502 is on canister 118 and coupling segment 504, 600 is on control unit housing 120. Further, the number, length, angle, and size of bayonet lug(s) 508 of coupling segment 502 may vary and accordingly, retaining slot 510 of coupling segment 504 will correspond to the size, number, and configuration of projection(s) 508 so that control unit housing 120 and canister 118 may be releasably joined together.

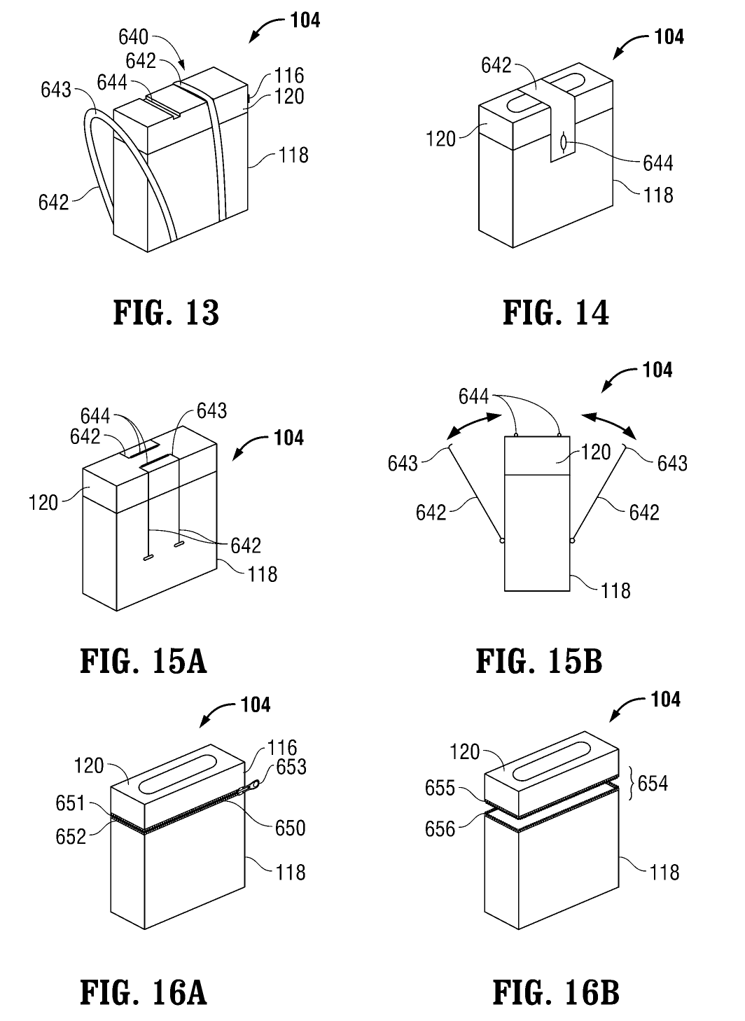

Referring now to FIG. 13 another embodiment of releasably connecting collection canister 118 and control unit housing 120 of subatmospheric pressure mechanism 104 is illustrated. Fastener mechanism 640 includes strap 642 and catch 644. Strap 642 may be of a predetermined length sufficient to wrap around housing 120 and firmly hold housing 120 to canister 118. Strap 642 may also be made of material having an elastic component in order for strap 642 to stretch around housing 120 and join the two components. In the alternative, strap 642 may be substantially rigid and formed of steel, titanium, a polymeric material or the like. Strap 642 includes closed looped end 643. Looped end 643 is passed around canister 118 and housing 120 to securely and firmly connect housing 120 to canister 118. Catch 644 are used to secure strap 642 to housing 120. In the current embodiment, catch 644 is in the form of indentations substantially parallel to the path of strap 642 for holding strap 642 on housing 120. To release, the operator pulls strap 642 away from housing 120. In embodiments, strap 642 has sufficient elasticity that it may catch on housing 120 without the use of catch 644.

FIG. 14 illustrates another embodiment of canister 118 having strap 642 and catch 644. Strap 642 is affixed to a side of canister 118 and catch 644 is disposed on an opposite side of canister 118. Catch 644 may be a button, knob, hook, clasp, bar, or other protrusion capable of securing strap 642. In embodiments, catch 644 may be one half of a fixation element such as a hook and loop fastener. To join housing 120 and canister 118, the operator passes strap 642 over housing 120 and secures it with catch 644.

FIGS. 15A and 15B illustrate another embodiment of canister 118 having an alternative placement of straps 642 and an alternate catch 644 than FIGS. 13 and 14. Canister 118 includes strap 642 anchored on canister 118 to form closed looped end 643. Housing 120 includes catch 644 in the form of a bar, but other protrusions as described above may be used. Strap 642 is placed around catch 644 thus preventing strap 642 from moving and releasably connecting canister 118 with housing 120. The operator may then release housing 120 by freeing strap 642 from catch 644.

In other embodiments, the fastener mechanism may include complementary joining members disposed around the periphery of housing 120 and canister 118, examples of which are shown in FIGS. 16-21. FIG. 16A illustrates housing 120 of control unit 116 and collection canister 118 releasably connected by zipper 650. Zipper 650 includes first teeth assembly 651, second teeth assembly 652, and slider 653. First and second teeth assemblies 651 and 652 are disposed on housing 120 and canister 118, respectively. Slider 653 may be attached to either first or second teeth assemblies 651 or 652. As known to those skilled in the art, slider 653 includes wedges that combine the hooks and hollows of first and second teeth assemblies 651 and 652 in order to fasten first and second teeth assemblies 651 and 652 together. FIG. 16B illustrates use of hook and loop fastener 654. Fastener 654 includes hook 655 and loop 656 material, each disposed on one of housing 120 and canister 118. As housing 120 is brought in contact with canister 118, the pressure exerted causes hooks 655 to entangle loops 656 to form a bond strong enough to hold housing 120 and canister 118 together. By exerting pressure to separate housing 120 from canister 118, hook and loop bonds are broken a few at a time along the length of fastener 654 in order to remove housing 120 from canister 118.

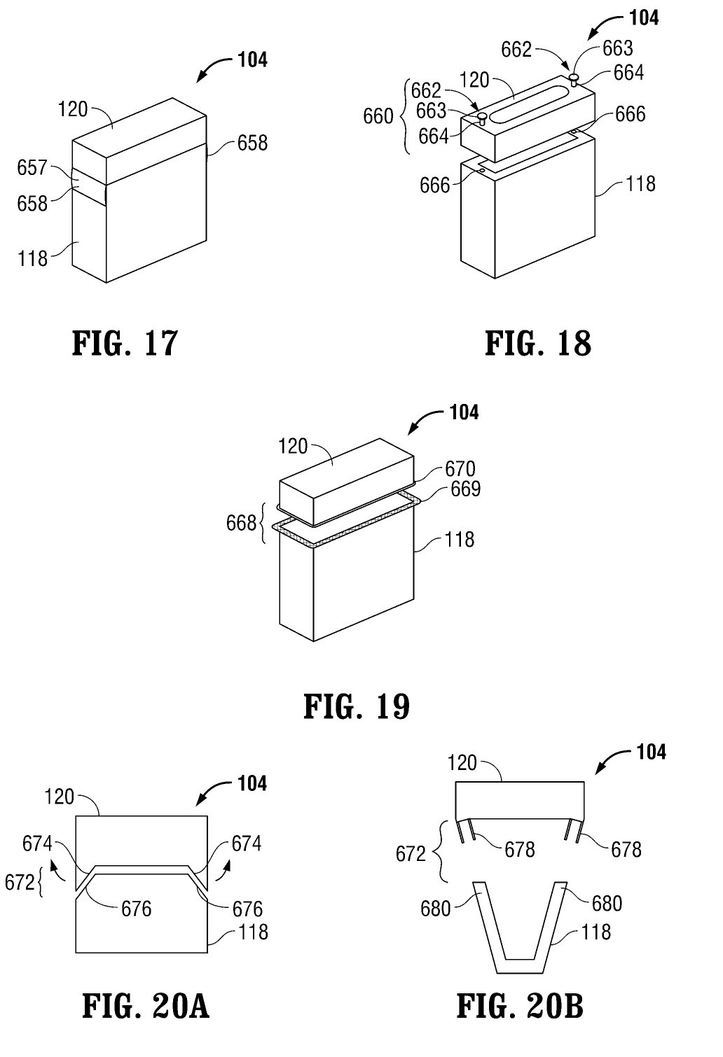

FIG. 17 illustrates use of door fastener 658. Door fastener 658 includes door 657. Door 657 is unfastened so that housing 120 may be placed on top of canister 118. Once placed together, door 657 is closed and locked in place. In embodiments, door 657 is located on canister 118. In other embodiments, door 657 is located on housing 120. It is envisioned that more than one door 657 may be used to securely engage canister 118 with housing 120.

FIG. 18 illustrates use of screw fastener 660. Screw fastener 660 includes thumbscrews 662 through housing 120 and receiving slots 666 in canister 118. Thumbscrew 662 includes gripping head 663 and threaded body 664. Head 663 may include ridged sides 665 for engaging the operator's fingers for better control of thumbscrew 662. The operator twists head 663 of thumbscrew 662 until threaded body 664 is firmly interlocked with slot 666 of canister 118. To release, thumbscrew 662 is twisted in the opposite direction. In embodiments, head 663 of thumbscrew 662 may be slotted for receiving a screwdriver.

FIG. 19 illustrates use of ring fastener 668. Ring 669 is disposed around canister 118 and gasket 670 on housing 120. Ring 669 may be fixed at one end to canister 118 or may be independently associated with canister 118. As housing 120 is placed on canister 118 gasket 670 deforms and compresses. Ring 669 may then be slidingly engaged around both housing 120 and canister 118 utilizing gasket 670 as a securement point. In embodiments, ring fastener 668 is constructed in reverse so that sliding ring 669 is disposed on housing 120 and gasket 670 on canister 118.

FIGS. 20A and 20B illustrate use of friction fit fastener 672. FIG. 20A illustrates housing 120 having angled edges 674 and canister 118 having complementary angled edges 676. As housing 120 is placed on canister 118 angled edges 674 of housing 120 slide over angled edges 676 of canister 118 slightly displacing edges 674 causing edges 674 to grip edges 676, thus holding housing 120 and canister 118 together. In embodiments, a gasket may be used between housing 120 and canister 118 to aid in removably sealing the two components together. FIG. 20B utilizes groove 678 in housing 120 and tongue 680 on canister 118. Tongue 680 is sized comparably to groove 678 so that as tongue 680 is inserted into groove 678 they frictionally fit together thus securing housing 120 with canister 118. In embodiments, friction fit fastener 672 is constructed in reverse, such that tongue 680 is located on housing 120 and groove 678 in canister 118.

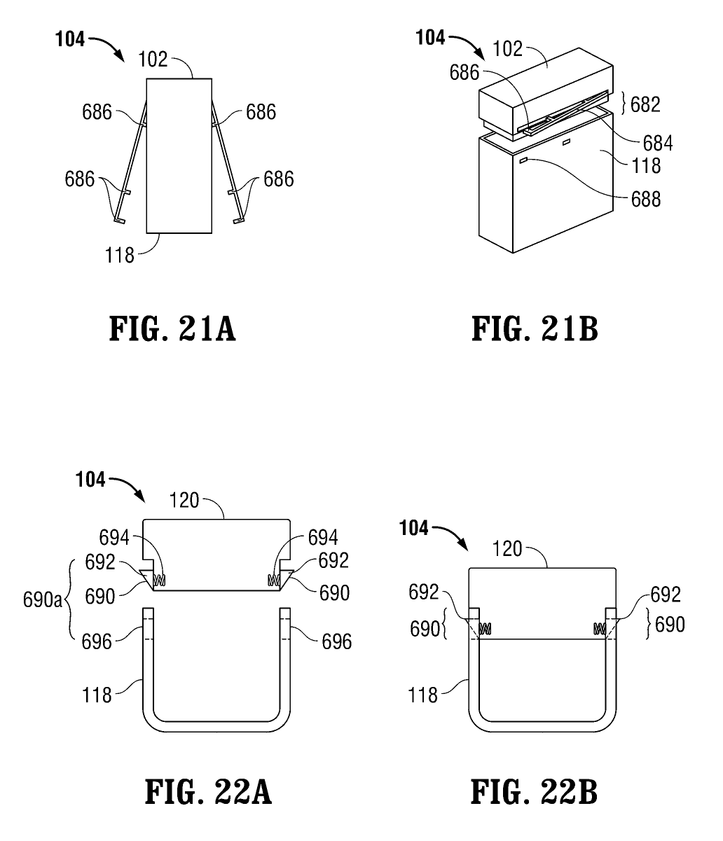

FIGS. 21A and 21D illustrate use of snap fastener 682 to facilitate connection between housing 120 and canister 118. Snap fastener 682 includes bar 684 having protrusions 686. One end of bar 684 is pivotably connected to housing 120. Indentations 688 are positioned on canister 118 such that when housing 120 is positioned on canister 118 and bar 684 pivoted towards canister 118, protrusions 686 and indentations 688 align and snap fit together thus joining canister 118 and housing 120. To release, the operator pulls bar 684 away from canister 118 thus disengaging protrusions 686 from indentations 688. In embodiments, snap fastener 682 is constructed in reverse, such that indentations 688 are located on housing 120 and bar 684 on canister 118.

In embodiments, the fastener mechanism comprises spring-loaded button fastener 690. Examples are included in FIGS. 22-24. In FIGS. 22A and 22B, housing 120 includes spring-loaded buttons 690. Button 690 includes button 692 and spring 694. Spring-loaded button 690a is shown in its unbiased state, while button 690b is shown in a biased position. Button 692 generally lies at an angle with the top portion protruding farther out from housing 120 than the bottom portion so that as housing 120 slides onto canister 118, buttons 692 compress against canister 118, returning to their biased position when reaching opening 696 in canister 118. The slope of button 692 also prevents housing 120 from slidingly detaching from canister 118 unless the operator presses button 692 in towards canister 118. In embodiments, buttons 692 are placed on the longer sides of canister 118 close to one end so that the operator may press both buttons 692 with one hand.

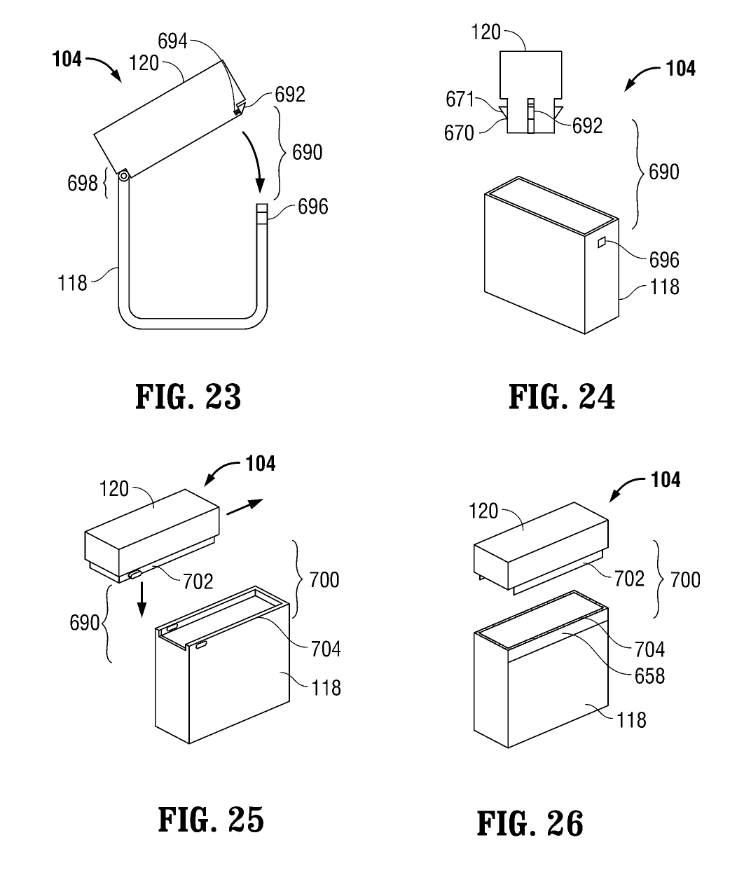

FIG. 23 illustrates another embodiment for fastening housing 120 and canister 118 using spring-loaded button fastener 690 and pivot point 698. Spring-loaded button fastener 690 is placed on one end of housing 120 and the other end is permanently or removably coupled to canister 118 at pivot point 698. The operator pivots housing 120 down towards canister 118 to engage spring-loaded button fastener 690. To disengage, the operator presses button 692 and pivots housing 120 back up. In embodiments, subatmospheric pressure mechanism 104 is a disposable unit. In other embodiments, canister 118 can be used with a disposable collection bag (not shown) placed therein. FIG. 24 illustrates another embodiment using spring-loaded button fastener 690. Gasket 670 is also utilized and includes grooved surface 671 which deforms and sticks down to canister 118 when housing 120 is placed on canister 118 and spring-loaded button fastener 690 is engaged.

In yet other embodiments, as illustrated in FIGS. 25 and 26, the fastener mechanism includes sliding mechanism 700. Sliding mechanism includes rail 702 and track 704, one of each being disposed on housing 120 and canister 118. Housing 120 and canister 118 are engaged by placing one end of housing 120 adjacent one end of canister 118 such that rail 702 and track 704 align. Housing 120 may then slide across canister 118 to a closed position. An additional fastening mechanism may be used in conjunction with mechanism 700, such as spring-loaded buttons 690 as illustrated in FIG. 25 and door 658 in FIG. 26.

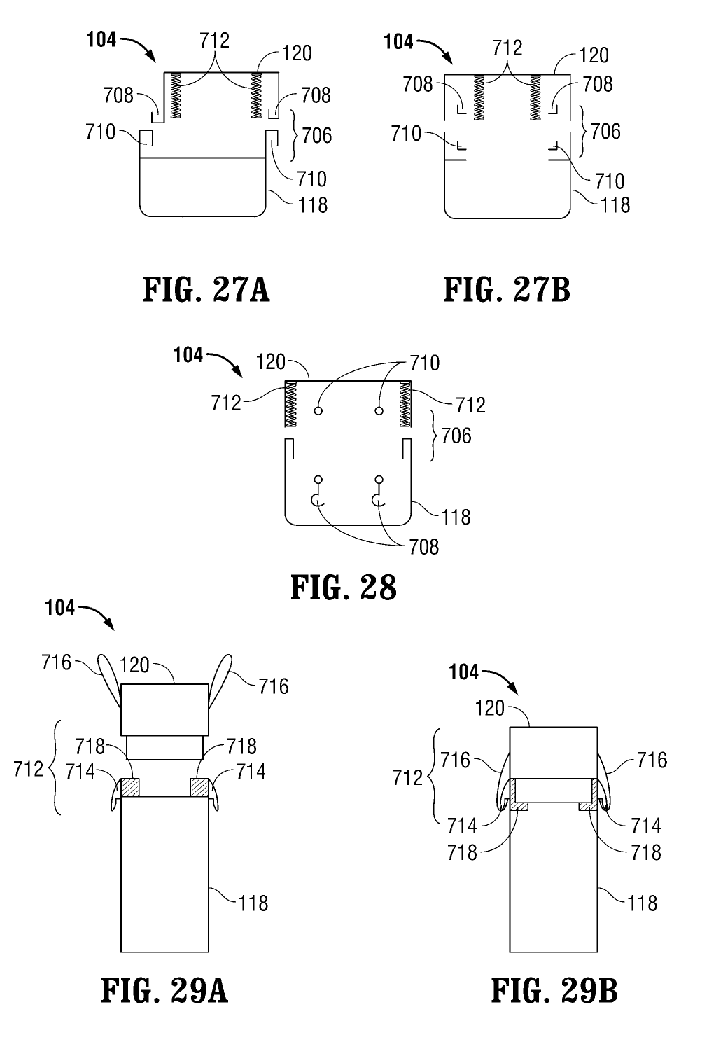

In other embodiments, the fastener mechanism may include spring-assisted clips 706. Spring-assisted clips 706 include hook element 708, grab element 710, and springs 712. FIGS. 27A and 27B illustrate alternate embodiments utilizing clips 706. Hook element 708 is disposed on housing 120 and springs 712 are placed adjacent to each hook element 708. Grab element 710 is located on canister 118 and is complementary to hook element 708. Housing 120 is placed on top of canister 118 and pressed down, so that springs 712 compress against canister 118. Housing 120 is slid onto canister 118 so that hook element 708 catches grab element 710. As the operator releases pressure on housing 120, springs 712 spring up and housing 120 remains connected to canister 118. To disconnect, the operator presses back down on housing 120 so that hook and grab elements 708 and 710 disconnect. The operator then slides housing 120 away from canister 118. Other embodiments, such as FIG. 28, illustrate alternate placement and types of hook and grab elements 708 and 710, respectively.

FIGS. 29A and 29B illustrate use of movable clip fastener 712 to releasably join housing 120 with canister 118. Clip fastener 712 includes fixed grab element 714, movable hook element 716, and gasket 718. Housing 120 includes movable hook element 716 and canister 118 includes fixed grab element 714 and gasket 718. As housing 120 is placed on canister 118, gasket 718 deforms and seals canister 118. Movable hook elements 716 are brought down towards canister 118 and hooked on fixed grab elements 714 thus connecting housing 120 and canister 118. To release, the operator releases movable hook element 716 from fixed grab element 714. The operator may also first press down on housing 120 to compress gasket 718 thus creating room to release movable hook element 716 from fixed grab element 714.

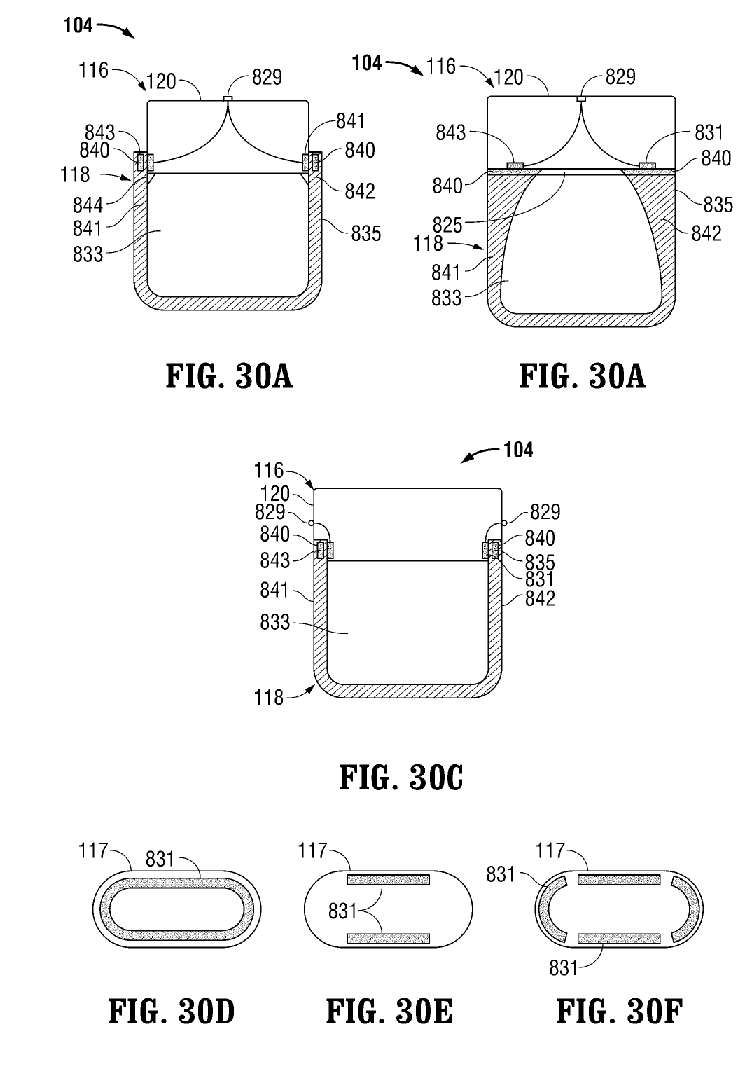

Referring now to FIGS. 30A-30C, other embodiments of releasably connecting collection canister 118 and control unit housing 120 are illustrated. Housing 120 includes switch 829 and at least one electromagnet 831 disposed along the periphery of housing 120. Switch 829 may be a button, knob, dial, or other adjuster capable of activating and/or de-activating electromagnet 831 by way of control circuitry (not shown). Switch 829 may be manually actuated, or, in the alternative, may be automatically actuated upon mounting of some of the components of the subatmospheric pressure mechanisms 104. The control circuitry recognizes changes in state of switch 829 and activates or de-activates the magnetic field of electromagnet 831 by altering the flow of current from a power source 168 (not shown). The magnetic field of electromagnet 831 can be manipulated over a wide range by controlling the amount of electric current from the power source.

Collection canister 118 includes magnetic or ferromagnetic material which may be disposed within or adjacent to upper portion 835 of canister 118. Magnetic material 840 may be a permanent magnet or metal capable of being attracted by the magnetic field created by electromagnet 831 of housing 120 of control unit 116. Such materials include nickel, iron, cobalt, their alloys, and the like. Other materials, within the purview of those skilled in the art, containing detectable magnetic properties may be used. Magnetic material 840 may be placed anywhere on canister 118. For example, FIG. 30A shows magnet material 840 in the form of an annular magnet disposed within the side walls 841 and 842 of canister 118. FIG. 30B shows magnetic material 840 disposed on top wall 843. FIG. 30C shows upper portion 835 of canister 118 made of magnetic material 840 and thus, a portion of canister 118 itself is magnet 840.

Housing 120 of control unit 116 is releasably connected to collection canister 118. Housing 120 of control unit 116 may be placed partially within canister 118 such as shown in FIG. 30A. Control unit housing 120 is supported by supports 844 at least partially disposed within canister 118. Control unit housing 120 may also be placed on top of canister 118 as shown in FIG. 30B. FIG. 30B also contains a lip or seal 825 to aid in aligning housing 120 of control unit 116 on canister 118 and to tightly close or seal canister 118.

When housing 120 of control unit 116 and canister 118 are mounted to each other, electromagnet 831 of control unit housing 120 and magnetic material 840 of canister 118 are substantially aligned or positioned such that magnets 831 and 840 are capable of attracting each other. Switch 829 may be indented within housing 120 to prevent inadvertent activation. In embodiments, such as FIG. 30C, two switches 829 are utilized thus making it more difficult to mistakenly engage both switches 829 and disconnect the components. Any number of magnets may be disposed around housing 120 of control unit 116 and controlled by switch 829, such as the configurations shown in FIGS. 30D-30F. As indicated hereinabove, switch 829 may be manually activated and may, e.g., be mounted to the exterior of either housing 120 or canister 118.

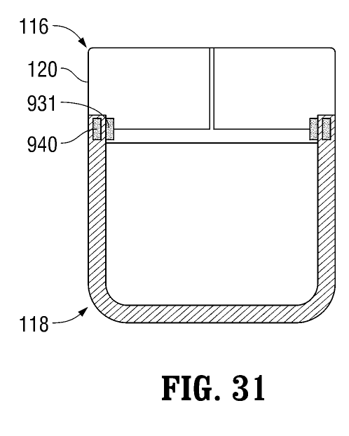

Alternatively, FIG. 31 illustrates use of a permanent magnet 931 instead of an electromagnet in control unit housing 120. The distance created between magnets 931 and 940 or change in dipole moments breaks the magnetic field and frees control unit housing 120 from canister 118.

To assemble the control unit housing 120 and the canister 118, the housing 120 and canister 118 are aligned and joined together by activating the magnetic field of electromagnet 831 by switch 829 of FIGS. 30A-30C (or the magnetic field of permanent magnets 931 and 940 of FIG. 31). Alternatively, housing 120 of control unit 116 and canister 118 may have complimentary sensors for detecting attachment which in turn signal control circuitry to activate a power source. Control unit housing 120 and canister 118 remain coupled until switch 829 is triggered and electromagnet 831 de-activated.

While the disclosure has been illustrated and described, it is not intended to be limited to the details shown, since various modifications and substitutions can be made without departing in any way from the spirit of the present disclosure. As such, further modifications and equivalents of the invention herein disclosed can occur to persons skilled in the art, and all such modifications and equivalents are believed to be within the spirit and scope of the disclosure as defined by the following claims.

What is claimed is:

1. A portable system for subatmospheric pressure therapy in connection with healing a surgical wound, which comprises:
a wound dressing dimensioned for positioning relative to a wound bed of a subject; and
a subatmospheric pressure mechanism dimensioned to be carried or worn by the subject, the subatmospheric pressure mechanism including a housing having a control unit, a collection canister in fluid communication with the wound dressing for collecting exudates from the wound bed removed under subatmospheric pressure supplied by the control unit, and means for releasably connecting the housing and the canister.

2. The system according to claim 1, wherein the means for releasably connecting the housing and the canister include a latch coupling.

3. The system according to claim 2, wherein the latch coupling includes a first coupling segment and a second coupling segment, the first coupling segment including a locking surface and the second coupling segment including a locking slot for securely receiving the locking surface of the first coupling segment.

4. The system according to claim 1, wherein the means for releasably connecting the housing and the canister include a bayonet mount.

5. The system according to claim 4, including a spring mechanism for facilitating releasable securement of the housing and the canister.

6. The system according to claim 1, wherein the means for releasably connecting the housing and the canister include a fastener mechanism.

7. The system according to claim 6, wherein the fastener mechanism is selected from strap fasteners, zip fasteners, hook and loop fasteners, door fasteners, screw fasteners, friction fit fasteners, snap fasteners, spring-loaded button fasteners, slide fasteners, and spring-assisted clip fasteners.

8. The system according to claim 1, wherein the means for releasably connecting the housing and the canister include magnetic coupling.

9. The system according to claim 8, including a switch for facilitating releasable electromagnetic securement of the housing and the canister.
